(19) United States
(12) Patent Application Publication
(10) Pub. No.: US 2015/0073456 A1
(43) Pub. Date: Mar. 12, 2015

(54) LIQUID INJECTION APPARATUS AND MEDICAL INSTRUMENT

(71) Applicant: SEIKO EPSON CORPORATION, Tokyo (JP)

(72) Inventors: Hirokazu SEKINO, Chino-shi (JP); Hideki KOJIMA, Matsumoto-shi (JP); Kazuaki UCHIDA, Fujimi-machi (JP)

(21) Appl. No.: 14/484,098

(22) Filed: Sep. 11, 2014

(30) Foreign Application Priority Data
Sep. 11, 2013 (JP) 2013-188259

(51) Int. Cl. A61B 17/3203

(52) U.S. Cl. CPC A61B 17/3203; A61B 2017/32032; A61B 2017/00973
USPC 606/167

(57) ABSTRACT

A liquid injection apparatus which includes a varying portion configured to vary a pressure in an interior of a liquid chamber in accordance with a drive signal, an injection tube having an injection port configured to inject liquid from the liquid chamber, a liquid supply unit configured to supply liquid to the liquid chamber, and a control unit configured to adjust the pressure in an interior of the liquid chamber by controlling the varying portion and the liquid supply unit. The control unit changes a rise time, which is a time period required for a drive signal to reach a second predetermined voltage from a first predetermined voltage in accordance with a speed of movement of the injection port.

### PRIORITY INFORMATION

The present invention claims priority to Japanese Patent Application No. 2013-188259 filed Sep. 11, 2013, which is incorporated herein by reference in its entirety.

### BACKGROUND

1. Technical Field

The present invention relates to injection of liquid.

2. Related Art

In a liquid injection apparatus used as a medical instrument, various methods of measuring acceleration at an injection port and selecting a mode of liquid injection on the basis of the acceleration are known. One such example is found in Japanese Patent Application No. JP-A-2012-143374.

A problem with this and other methods is that the depth of resection cannot be stabilized due to variations in speed of movement of the injection port.

### SUMMARY

An advantage of some aspects of the invention is to solve at least part of the problem described above, and the invention can be implemented as the following forms.

An aspect of the invention provides a liquid injection apparatus. The liquid injection apparatus includes a varying portion configured to vary a pressure in the interior of a liquid chamber in accordance with a drive signal, an injection tube having an injection port configured to inject liquid from the liquid chamber, a liquid supply unit configured to supply liquid to the liquid chamber, and a control unit configured to adjust the pressure in the interior of the liquid chamber by controlling the varying portion and the liquid supply unit, wherein the control unit changes a time period required for the drive signal to reach a second predetermined voltage from a first predetermined voltage in accordance with a speed of movement of the injection port. According to this aspect, since a rise time (the time period required for the drive signal to reach the second predetermined voltage from the first predetermined voltage) is changed in accordance with the speed of movement, the depth of resection is stabilized because the rise time is a parameter related to the depth of resection.

The invention may be implemented in various forms other than those described above. For example, the invention may be implemented in a form such as a method of injecting liquid, a method of surgical operation, programs for implementing these methods, a storage medium having these programs stored therein.

### BRIEF DESCRIPTION OF DRAWINGS

The invention will be described with reference to the accompanying drawings, wherein like numbers reference like elements.

FIG. 1 is a configuration drawing of a liquid injection apparatus.

FIG. 2 is a structural drawing illustrating an interior of the liquid injection mechanism.

FIG. 3 is a flowchart showing an injecting process.

FIG. 4 is a graph showing a waveform corresponding to one cycle of a drive waveform.

FIG. 5 is a graph showing a relationship between a rise time and a speed of an injection port.

FIG. 6 is a graph showing a relationship between a peak voltage and the speed of an injection port.

FIG. 7 is a graph showing a method of determination of a flow amount.

FIG. 8 is a table showing a result of experiment in which an influence of variations in rise time were inspected.

### DESCRIPTION OF EXEMPLARY EMBODIMENTS

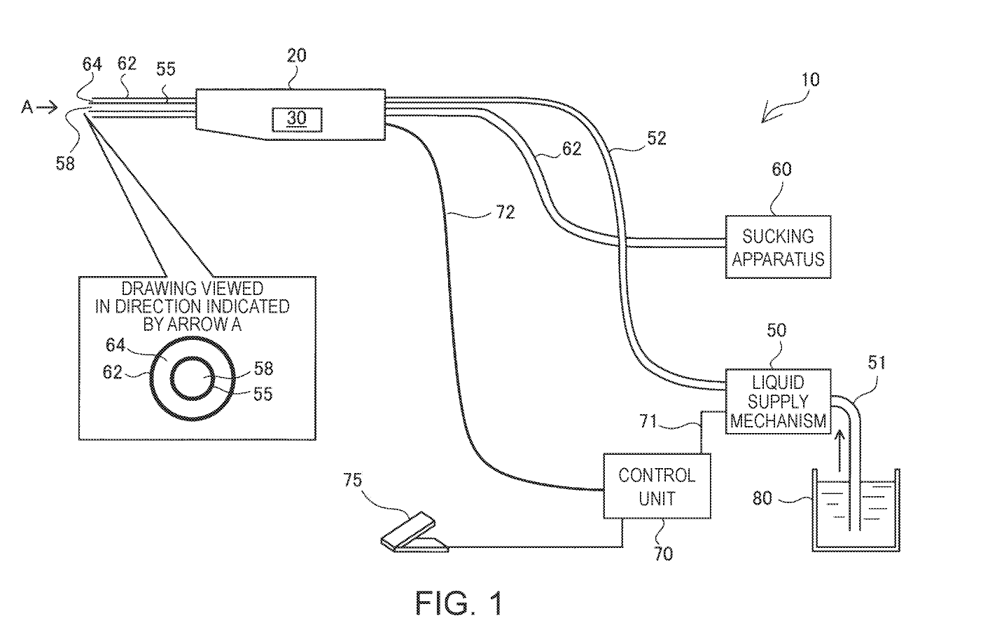

FIG. 1 illustrates a configuration of a liquid injection apparatus 10. The liquid injection apparatus 10 is a medical instrument used in a medical organization, and has a function to incise and resect an affected area by injecting liquid toward the affected area.

The liquid injection apparatus 10 includes a liquid injection mechanism 20, a liquid supply mechanism 50, a sucking apparatus 60, a control unit 70, and a liquid container 80. The liquid supply mechanism 50 and the liquid container 80 are connected to each other by a connecting tube 51. The liquid supply mechanism 50 and the liquid injection mechanism 20 are connected to each other by a liquid supply flow channel 52. The connecting tube 51 and the liquid supply flow channel 52 are formed of a resin. The connecting tube 51 and the liquid supply flow channel 52 may be formed of a material other than the resin (a metal, for example).

The liquid container 80 stores normal saline solution. The liquid may be a pure ware or a drug solution instead of the normal saline solution. The liquid supply mechanism 50 supplies liquid sucked from the liquid container 80 via the connecting tube 51 to the liquid injection mechanism 20 via the liquid supply flow channel 52.

The liquid injection mechanism 20 is an instrument that a user of the liquid injection apparatus 10 operates by holding in his or her hand. The user performs incision or resection of an affected area by injecting the liquid injected intermittently from an injection port 58 onto the affected area.

The control unit 70 sends a drive signal to a pulsation generating unit 30 via a signal cable 72. The control unit 70 controls a flow amount of liquid supplied to the pulsation generating unit 30 by controlling the liquid supply mechanism 50 via a control cable 71. A foot switch 75 is connected to the control unit 70. When the user turns the foot switch 75 ON, the control unit 70 controls the liquid supply mechanism 50 to cause the pulsation generating unit 30 to supply liquid and sends the drive signal to the pulsation generating unit 30 to cause the pressure of the liquid supplied to the pulsation generating unit 30 to generate pulsation.

The sucking apparatus 60 is used to suck liquid or resected tissue around the injection port 58. The sucking apparatus 60 and the liquid injection mechanism 20 are connected to each other by a sucking flow channel 62. The sucking apparatus 60 applies a negative pressure or a sucking force to an interior of the sucking flow channel 62 constantly while the switch is ON. The sucking flow channel 62 penetrates through an interior of the liquid injection mechanism 20 and opening in the vicinity of a distal end of an injection tube 55.

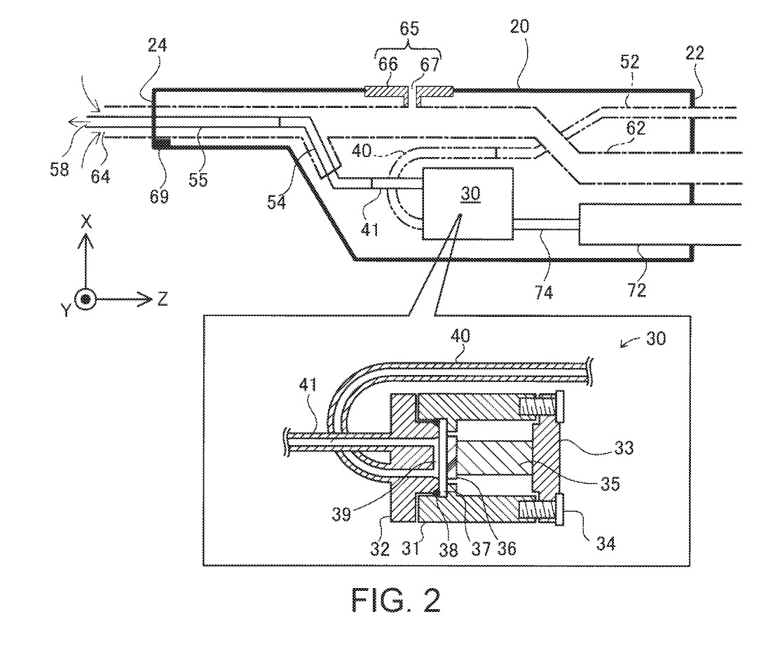

The sucking flow channel 62 lays over the injection tube 55 in the interior of the liquid injection mechanism 20, thereby forming a substantially concentric cylinder by a wall of the injection tube 55 and a wall of the sucking flow channel 62 as illustrated in a drawing viewed in an direction indicated by an arrow A in FIG. 1. A flow channel in which a sucked material sucked from a suction port 64 which corresponds to the distal end of the sucking flow channel 62 flows is defined between an outer wall of the injection tube 55 and an inner wall of the sucking flow channel 62. The sucked material is sucked to the sucking apparatus 60 via the sucking flow channel 62. The suction is adjusted by a suction applying mechanism, which will be described later, with reference to FIG. 2.

FIG. 2 is a structural drawing illustrating the interior of the liquid injection mechanism 20. The liquid injection mechanism 20 includes the pulsation generating unit 30, an inlet flow channel 40, an outlet flow channel 41, a connecting tube 54, and an acceleration sensor 69 integrated in the interior thereof, and is provided with a sucking force adjusting mechanism 65.

The pulsation generating unit 30 generates pulsation in the pressure of liquid supplied from the liquid supply mechanism 50 to the liquid injection mechanism 20 via the liquid supply flow channel 52. The pressurized and pulsed liquid is supplied to the injection tube 55. The liquid supplied to the injection tube 55 is injected intermittently from the injection port 58. The injection tube 55 is formed of stainless steel. The injection tube 55 may be formed of other materials having a predetermined or more rigidity such as other metals, for example, brass, or a reinforced plastic.

The pulsation generating unit 30 includes a first case 31, a second case 32, a third case 33, a bolt 34, a piezoelectric element 35, a reinforcing plate 36, a diaphragm 37, a packing 38, the inlet flow channel 40, and the outlet flow channel 41 as illustrated in a lower portion in FIG. 2. The first case 31 and the second case 32 are joined so as to oppose each other. The first case 31 is a cylindrical member. One end of the first case 31 is sealed by fixing the third case 33 with the bolt 34. A piezoelectric element 35 is arranged in a space defined in an interior of the first case 31.

The piezoelectric element 35 is a multi-layer piezoelectric element. One end of the piezoelectric element 35 is secured to the diaphragm 37 via the reinforcing plate 36. The other end of the piezoelectric element 35 is secured to the third case 33. The diaphragm 37 is formed of a metallic thin film, and is secured to the first case 31 at a peripheral edge portion thereof. A liquid chamber 39 is formed between the diaphragm 37 and the second case 32. A capacity of the liquid chamber 39 is varied by driving the piezoelectric element 35.

The signal cable 72 is inserted from a rear end portion 22 of the liquid injection mechanism 20. Two electrode lines 74 are stored in the signal cable 72, and are connected to the piezoelectric element 35 in an interior of the pulsation generating unit 30. A drive signal transmitted from the control unit 70 is sent to the piezoelectric element 35 via the electrode lines 74 in an interior of the signal cable 72. The piezoelectric element 35 expands and contracts on the basis of the drive signal.

The inlet flow channel 40 through which the liquid flows is connected to the second case 32. The inlet flow channel 40 is bent into a U-shape and extends toward the rear end portion 22 of the liquid injection mechanism 20. The liquid supply flow channel 52 is connected to the inlet flow channel 40. The liquid supplied from the liquid supply mechanism 50 is supplied to the liquid chamber 39 via the liquid supply flow channel 52.

When the piezoelectric element 35 expands and contracts at a predetermined frequency, the diaphragm 37 vibrates. When the diaphragm 37 vibrates, the capacity of the liquid chamber 39 varies, and hence the pressure of the liquid in an interior of the liquid chamber pulsates. The liquid passed through the liquid chamber 39 flows out from the outlet flow channel 41.

The outlet flow channel 41 is connected to the second case 32. The injection tube 55 is connected to the outlet flow channel 41 via the metallic connecting tube 54. The liquid flowing out into the outlet flow channel 41 is injected from the injection port 58 via the connecting tube 54 and the injection tube 55.

The sucking force adjusting mechanism 65 is configured to adjust a force of the sucking flow channel 62 for sucking liquid or the like from the suction port 64. The suction force adjusting mechanism 65 includes an operating portion 66 and a hole 67. The hole 67 is a through hole for connecting the sucking flow channel 62 and the operating portion 66. When the user opens and closes the hole 67 with a finger of his or her hand gripping the liquid injection mechanism 20, the amount of air flowing into the sucking flow channel 62 through the hole 67 is adjusted by the extent of opening and closing of the hole 67, and hence a suction force of the suction port 64 is adjusted. The adjustment of the suction force is realized by being controlled by the sucking apparatus 60.

The liquid injection mechanism 20 is provided with the acceleration sensor 69. The acceleration sensor 69 is a piezoresistive three-axis accelerator sensor. The three axes correspond to respective axes of XYZ illustrated in FIG. 2. The X-axis is parallel to a direction of penetration of the hole 67, and an upper direction corresponds to a positive direction. The Z-axis is parallel to a direction of a longitudinal axis of the injection tube 55, and a direction in which the liquid is injected corresponds to a negative direction. The Y-axis is defined by a right hand system with reference to the X-axis and the Z-axis.

The acceleration sensor 69 is arranged in the vicinity of a distal end portion 24 as illustrated in FIG. 2. The result of measurement is input to the control unit 70 via the signal line (not illustrated) and the signal cable 72.

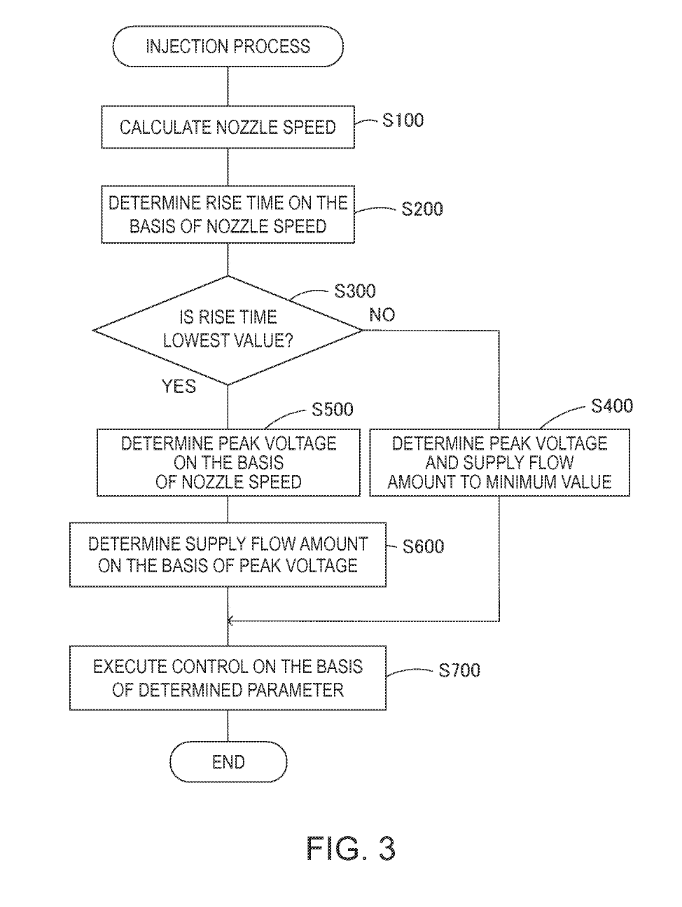

FIG. 3 is a flowchart showing an injecting process. The injection process is repeatedly executed by the control unit 70 while the foot switch 75 is pressed downward. First of all, a speed S of the injection port 58 is calculated (Step s100). The speed S here is an absolute value of the speed on an XY plane. In other words, it is an absolute value of the speed with the speed in a Z-axis direction ignored. The speed S is calculated on the basis of the acceleration in three-axis measured by the acceleration sensor 69.

The speed S is calculated as a parameter which affects the depth of resection of the affected area. The reason is that a resection performance acting per unit time on the respective local region of the affected area is affected by a relative speed between the injection port 58 and the affected area. In the embodiment, although the speed S may be handled as a relative speed between the affected area and the injection port 58 considering the case where the affected area moves in association with aspiration of the patient, description will be given on the assumption that movement of the affected area stays in a state to be not more than a predetermined amount of movement.

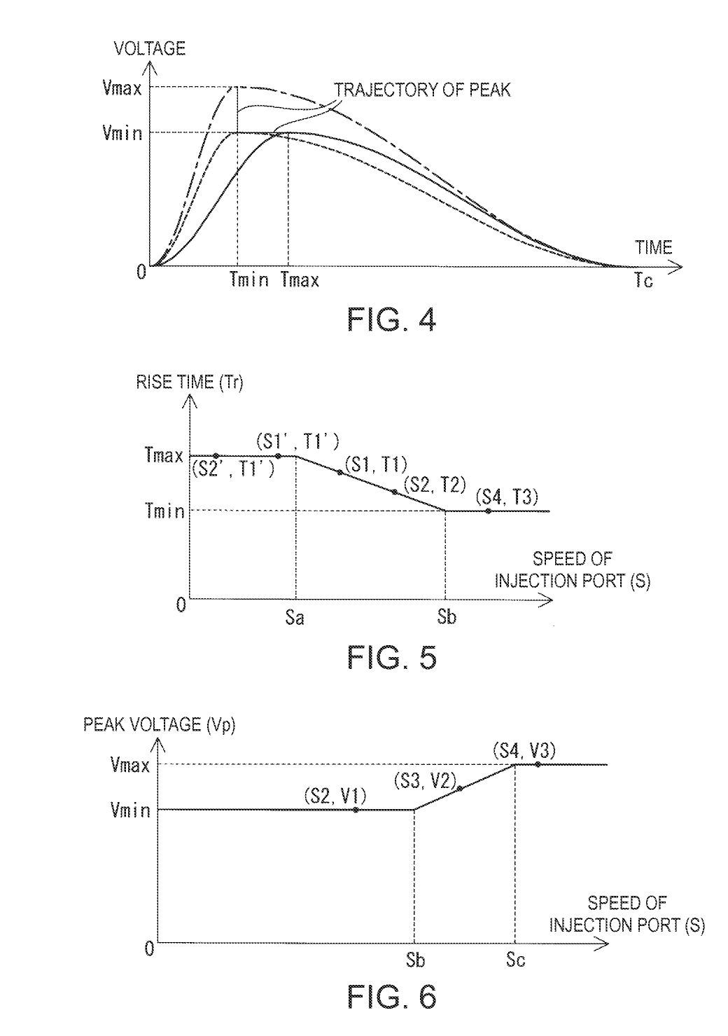

Subsequently, a rise time having a waveform of a drive signal (hereinafter, referred to as “drive waveform”) is determined on the basis of the calculated speed S (Step 200). FIG. 4 is a graph showing a waveform corresponding to one cycle of a drive waveform. A vertical axis represents voltage, and a lateral axis represents time.

The drive waveform of the embodiment is described as a combination of sine curves. The voltage from zero to a peak value is described by the following expression.

V(T)=Vp{1−cos(πT/Tr)}/2 (where 0≦T≦Tr)

In the expression, V is a voltage, Vp is a voltage peak value (peak voltage), T is a time period, and Tr is a rise time. Vp is a variable value set in a range of Vmin≦Vp≦Vmax. Tr is a variable value set within a range of Tmin≦Tr≦Tmax. Values of Vmax and Tmin are values predetermined on the conditions that the load of the piezoelectric element 35 or the like does not become too large. The values Vmin and Tmax are values predetermined on the conditions that the liquid is injected intermittently. The peak voltage indicates the maximum voltage in one cycle of the drive waveform used when injecting the liquid.

The voltage from the peak voltage to zero is described by the following expression.

V(T)=Vp[1+cos{π(T−Tr)/(Tc−Tr)}]/2 (where Tr≦T≦Tc)

The value Tc is a time period for one cycle of the drive waveform, and is a fixed value in the embodiment. As is clear from the above-described two expressions, the rise time Tr corresponds to a time period from the predetermined voltage in one cycle of the drive waveform to the peak of the voltage.

When the voltage of the drive signal is increased, the piezoelectric element 35 is deformed so that the capacity of the liquid chamber 39 contracts. When the rise time Tr is reduced, the contraction of the liquid chamber 39 is executed in a short time. Consequently, the liquid jets out, the resection performance is enhanced, and the depth of resection is increased.

FIG. 5 is a graph showing a relationship between the rise time Tr and the speed S in the embodiment. As shown in FIG. 5, in the case of S≦Sa, the rise time Tr is fixed to Tmax. In the case of Sa≦S≦Sb, the rise time Tr is linearly reduced in association with the increase in speed S. In the case of S≧Sb, the rise time Tr is fixed to Tmin. In Step S200, the rise time Tr is determined in accordance with the relationship shown above.

Subsequently, whether the rise time Tr is set to the lowest value (Tmin) is determined (Step S300). When the rise time Tr is determined to be a value other than the lowest value (No in Step 300), the peak voltage Vp and a supply flow amount are set to minimum values (Vmin) (Step S400).

In contrast, when the rise time Tr is determined to be the lowest value (Yes in Step S300), the peak voltage Vp of the drive signal is determined on the basis of the speed S (Step S500).

FIG. 6 is a graph showing a relationship between the peak voltage Vp and the speed S in the embodiment. As shown in FIG. 6, in the case of S≦Sb, the peak voltage Vp is fixed to Vmin. In order to realize such a relationship, if the rise time Tr is not the lowest value, the peak voltage Vp is fixed to Vmin as described above.

In the case of Sb≦S≦Sc, the peak voltage Vp is linearly increased in association with the increase in speed S. In the case of S≧Sc, the peak voltage Vp is fixed to Vmax. In the case where Step S500 is executed, since the relation S≧Sb is established, the peak voltage Vp is determined in accordance with the relationship with respect to the peak voltage Vp in this speed range.

Since the rise time Tr and the peak voltage Vp are determined as described above, the peak of the drive waveform follows an L-shaped trajectory as shown in FIG. 4.

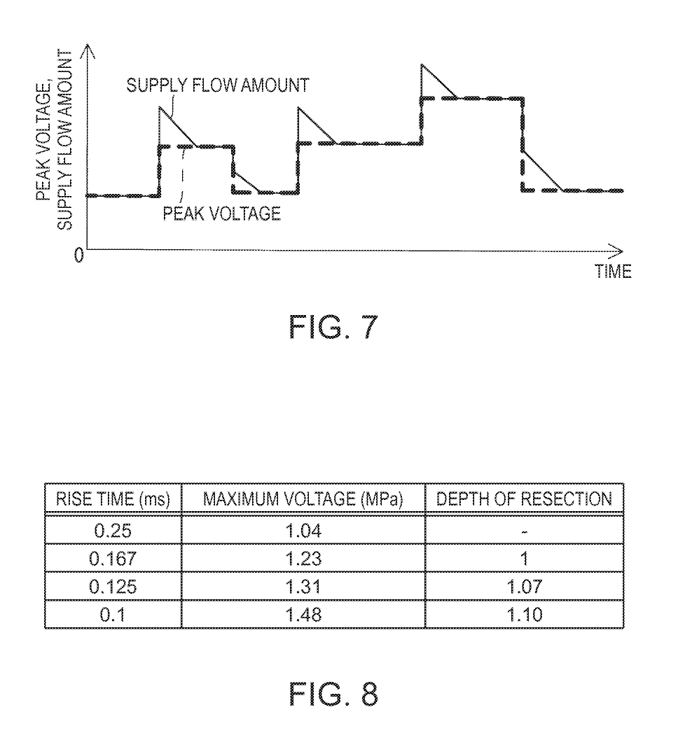

Subsequently, the supply flow amount is determined on the basis of the peak voltage Vp (Step S600). FIG. 7 is a graph showing a method of determination of the flow amount. A vertical axis represents the peak voltage Vp and the supply flow amount, and a lateral axis represents time. A change rate of the supply flow amount may be aligned with a change rate of the peak voltage. However, when the peak voltage changes, the supply flow amount is temporarily increased.

For example, when the state in which the peak voltage reaches Vp1 and the supply flow amount is F1 is changed to the state in which the peak voltage is 2×Vp1, the supply flow amount is increased temporarily to 3×F1, and then is reduced gradually to 2×F1. Alternatively, in the case where the state in which the peak voltage is Vp1 and the supply flow amount is F1 to the state in which the peak voltage is 0.5×Vp1, the supply flow amount is increased temporarily to 0.75×F1 and then is reduced gradually to 0.5×F1.

In this manner, when the peak voltage is changed, the supply flow amount is temporarily increased to avoid an event that the supply flow amount runs short and hence the injection of liquid cannot be executed normally.

Finally, control is executed on the basis of the determined parameters (the rise time Tr, the peak voltage Vp, and the supply flow amount) (Step S700). Consequently, the liquid is injected intermittently from the injection port 58 in accordance with the speed of the injection port 58.

FIG. 8 is a table showing a result of experiment in which a relationship among the rise time, the maximum voltage of the liquid to be injected, and the change of the depth of resection was inspected. The depth of resection was with reference to the case where the rise time is 0.375 ms. Measurement of the depth of resection was performed under the same conditions other than the rise time. This experiment was conducted without moving the injection port 58.

As shown in FIG. 8, as the rise time is decreased, the maximum voltage of the liquid increases, and the depth of resection is increased. In contrast, when the speed S is increased, the resection performance that acts on the respective local regions of the affected area is lowered. Therefore, in the case where the speed S is increased, the depth of resection may be stabilized by reducing the rise time.

Furthermore, according to the embodiment, in the case where the speed S is not more than Sb, the peak voltage Vp is constant, and hence an excluded volume does not change, so that there is no necessity to vary the supply flow amount. As may be understood by one of skill in the art, by maintaining the excluded volume, control is facilitated.

The piezoelectric element 35 and the diaphragm 37 of the embodiment are examples of the varying portion in the appended claims. Values S1 to S4 shown in FIG. 5 and FIG. 6 are first to fourth speeds, T1 to T3 are first to third time periods, V1 to V3 are first to third voltages, S1′ and S2′ are first and second predetermined time periods, and T1′ is an example of a predetermined value.

The invention is not limited to the embodiments, examples, and modifications in this specification and may be implemented in various configurations without departing the scope of the invention. For example, technical characteristics in the embodiments, the examples, and the modifications corresponding to the technical characteristics in the respective embodiments in the respective aspects described in the paragraph of the summary may be replaced or combined as needed in order to solve part or entire problem described above or in order to achieve part or entire part of the above-described advantages. The technical characteristics may be eliminated as needed unless otherwise specified to be essential in the specification. For example, the followings are exemplified.

The rise time and the peak voltage maybe determined by using a function, or may be determined by mapping in advance and substituting the speed S into the map. According to map control, a processing load is alleviated.

The speed range in which the rise time is to be varied and the speed range in which the peak voltage is to be varied may overlap with each other.

The drive waveform may not be a combination of sine curves, and for example, may be increased or decreased stepwise.

The relationship between the rise time and the speed of the injection port may be defined in curve or stepwise.

The definition of the rise time may not be the time period required for the drive signal to reach the peak from zero and, for example, may be a time period required for the drive signal to reach a value slightly smaller than the peak from a value slightly larger than zero.

In the case where the volume of the liquid chamber is contracted in the case where the voltage of the drive signal is lowered, the rise time maybe defined as time period required for reaching from a certain voltage value to a value smaller than the voltage value.

The speed of the injection port may be calculated or detected by the acceleration sensor installed at the distal end of the injection port, for example. In this case, it is considered that the result of calculation becomes more accurate.

Alternatively, the speed of the injection port may be calculated by using an image processing. For example, the speed of the injection port may be calculated by installing a marker at the distal end of the injection port, and following a movement of the marker with a camera.

In the case where the robot operates the liquid injection apparatus, the speed of the injection port is not necessary to be calculated because the robot can detect the speed, and the value detected by the robot may be used.

The speed of movement of the injection port may be calculated by adding the speed of movement of the affected area. Measurement of the speed of movement of the affected area may be achieved by estimating or measuring the movement caused by aspiration or pulse beat.

Also, energy to be applied to the liquid in the interior of the liquid chamber in accordance with the speed of the injection port may be adjusted so that the same energy is applied to the respective unit areas of an injection object by the liquid injected from the liquid injection mechanism.

In the embodiment, the liquid injection mechanism 20 has been described as an instrument that the user operates by holding with his or her hand. However, the liquid injection mechanism 20 may be an instrument to be inserted into a biological body and operated therein as the liquid injection mechanism used in an endoscope such as abdominoscope.

The type of the acceleration sensor may be a capacitance type or a thermal detection type. The sensor is not limited to detect the acceleration, but may be a sensor capable of detecting the speed directly or indirectly.

The liquid injection apparatus may be used in applications other than the medical instrument. For example, the liquid injection apparatus may be used in a cleaning apparatus configured to clean the stain by injected liquid.

The liquid injection apparatus may be used in a drawing apparatus configured to draw a line with injected liquid.

What is claimed is:

1. A liquid injection apparatus comprising:
a varying portion configured to vary a pressure in an interior of a liquid chamber in accordance with a drive signal;
an injection tube having an injection port configured to inject liquid from the liquid chamber;
a liquid supply unit configured to supply liquid to the liquid chamber; and
a control unit configured to adjust the pressure in the interior of the liquid chamber by controlling the varying portion and the liquid supply unit, wherein
the control unit changes a time period required for the drive signal to reach a second predetermined voltage from a first predetermined voltage in accordance with a speed of movement of the injection port.

2. The liquid injection apparatus according to claim 1, wherein
the control unit sets a rise time which is a time period required for the drive signal to reach from the first predetermined voltage to the second predetermined voltage to a first time period in the case where the speed of movement is a first speed, and sets the rise time to a second time period which is shorter than the first time period in the case where the speed of movement is a second speed which is faster than the first speed.

3. The liquid injection apparatus according to claim 2, wherein
the control unit sets a maximum voltage of the drive signal to a first voltage in the case where the speed of movement is the second speed, and sets the maximum voltage of the drive signal to a second voltage which is higher than the first voltage in the case where the speed of movement is a third speed which is faster than the second speed.

4. The liquid injection apparatus according to claim 3, wherein
the control unit sets a flow amount of the liquid to a first flow amount in the case where the speed of movement is the second speed, and sets the flow amount of the liquid to a second flow amount which is larger than the first flow amount in the case where the speed of movement is the third speed.

5. The liquid injection apparatus according to claim 4, wherein
the control unit sets the rise time to a third time period which is shorter than the second time period in the case where the speed of movement is a fourth speed which is faster than the third speed, and sets the maximum voltage of the drive signal to a third voltage which is higher than the second voltage.

6. The liquid injection apparatus according to claim 5, wherein
the control unit sets the flow amount of the liquid to a third flow amount in the case where the speed of movement is the fourth speed, the third flow amount being greater than the first flow amount or the second flow amount.

7. The liquid injection apparatus according to claim 2, wherein
the control unit sets the rise time, the maximum voltage of the drive signal, and the flow amount of the liquid to respective predetermined values in the case where the speed of movement is a first predetermined speed which is slower than the first speed, and the case where the speed of movement is a second predetermined speed which is lower than the first predetermined speed.

8. A medical instrument comprising the liquid injection apparatus according to claim 1.

9. A medical instrument comprising the liquid injection apparatus according to claim 2.

10. A medical instrument comprising the liquid injection apparatus according to claim 3.

11. A medical instrument comprising the liquid injection apparatus according to claim 4.

12. A medical instrument comprising the liquid injection apparatus according to claim 5.

13. A medical instrument comprising the liquid injection apparatus according to claim 6.

14. A medical instrument comprising the liquid injection apparatus according to claim 7.
